(19) United States
(12) Patent Application Publication
(10) Pub. No.: US 2007/0270770 A1
(43) Pub. Date: Nov. 22, 2007

(54) Venous access port assembly and method of making same

(75) Inventor: Raymond Bizup, Feasterville, PA (US)

Correspondence Address: ANTON P. NESS;FOX ROTHSCHILD LLP, 1250 SOUTH BROAD STREET, SUITE 1000, P..O. BOX 431, LANSDALE, PA, 19446-0431, US

(73) Assignee: Medical Components, Inc., Harleysville, PA (US)

(21) Appl. No.: 11/801,050

(22) Filed: May 7, 2007

Related U.S. Application Data
(60) Provisional application No. 60/801,523, filed on May 18, 2006.

(51) Int. Cl. A61M 31/00

(52) U.S. Cl. 604/288.02

(57) ABSTRACT

A venous access port assembly (10) having a housing base (28) with a discharge port (16), a septum (14) and a cap (48). An interior reservoir (22) is defined by a well (30) in the housing base and a bottom (62) of the septum, and a passageway (20) extends from the reservoir through the discharge port (16). The cap (48) is secured to an upper portion of the housing base and securely retains the septum in the assembly (10), compressing an annular flange (60) of the septum against a septum seat (52) of the housing base. The cap (48) is mechanically secured to the housing base (28) as well as being bonded thereto. A method is disclosed for mechanically securing the cap (48) to the housing base (28), in a manner compressing the flange (60) of the septum (14) and sealing the reservoir (22).

### CROSS-REFERENCE TO RELATED APPLICATIONS

This application claims priority from U.S. Provisional Patent Application Ser. No. 60/801,523 filed May 18, 2006.

### FIELD OF THE INVENTION

This relates to the field of medical devices and more particularly to catheter assemblies and ports therefor, for the infusion of fluids into the patient and withdrawal of fluids from the patient.

### BACKGROUND OF THE INVENTION

Infusion ports for the infusion and/or withdrawal of fluids from a patient are well-known, secured to the proximal end of an implanted catheter. These ports are typically used for drug infusion or small amounts of blood withdrawal, where large flows of fluid are not required. The ports are assemblies of a needle-impenetrable housing with a discharge port in fluid communication with the catheter and the reservoir within the port housing, and provide a subcutaneous self-sealing septum that defines an access site for multiple needle sticks through the covering skin tissue of the patient, through the septum and into the reservoir, without the need to continuously search for new access sites. Examples of such ports are disclosed, for example, in U.S. Pat. Nos. 4,704,103; 4,762,517; 4,778,452; 5,185,003; 5,213,574 and 5,637,102.

It is desired to provide a venous access port assembly that is assuredly secured together in an assuredly sealed manner.

### BRIEF SUMMARY OF THE INVENTION

The present invention is a venous access port having a housing and a septum, providing an interior reservoir and a passageway extending from the reservoir through a stem of a discharge port to establish fluid communication with a proximal end of a catheter lumen to which the port assembly is secured prior to placement of the assembly into a patient. The housing includes a base and a cap that together cooperate to secure a needle-penetrable septum within the assembly by compressing a seating flange of the septum in a seat of the housing base. The cap is mechanically secured to the housing base by a mechanical joint, preferably a retention rib of one of the housing base and cap extending radially outwardly to be received into a retention groove along the inside surface of the other of the housing base and cap in a snap fit, extending around most of the circumference of the port assembly. Preferably, solvent bonding is also provided between adjacent surfaces of the housing base and cap.

In a first embodiment, the retention rib is defined on the housing base's exterior surface, with the retention groove defined on the cap's interior surface. In a second embodiment, the retention rib is defined on the cap's interior surface, with the retention groove defined on the housing base's exterior surface.

In the method of the present invention, a cap of a venous access port assembly is mechanically secured to a housing base of the venous access port assembly by retention sections. The retention sections mechanically secure the cap to the housing base and compress a flange of a septum during curing of a solvent that further secures the cap to the housing base without fixtures.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an isometric view of the venous access port of the present invention;

FIG. 2 is a plan view of the port of FIG. 1;

FIGS. 3 and 4 are cross-section views of the port of FIGS. 1 and 2 taken along lines 3-3 and lines 4-4 of FIG. 1, respectively;

FIG. 5 is an enlarged view of the port's discharge port taken along lines 5-5 of FIG. 4;

FIG. 6 is an isometric view of the needle-impenetrable housing base of the venous access port of FIG. 1;

FIG. 7 is a plan view of the housing base of FIG. 6;

FIGS. 8 and 9 are cross-sectional views of the housing of FIGS. 6 and 7 taken along lines 8-8 and 9-9 of FIG. 7;

FIGS. 10 and 11 are enlarged views taken along lines 10-10 and 11-11 of FIG. 9, respectively;

FIGS. 12 and 13 are side and cross-sectional views, respectively, of the septum of the venous access port of FIG. 1;

FIG. 14 is an enlarged view taken along lines 14-14 of FIG. 13;

FIGS. 15 to 17 are a plan view of the cap of the venous access port of FIG. 1, and cross-sectional views taken along lines 16-16 and 17-17 of FIG. 15, respectively; and

FIGS. 18 to 21 are views of a second embodiment of the venous access port assembly of the present invention, wherein FIGS. 18 and 19 are isometric views of the cap and housing base, respectively, FIG. 20 is a cross-sectional view of the cap, and FIG. 21 is a cross-sectional view of the assembly.

### DETAILED DESCRIPTION OF THE INVENTION

Certain terminology is used herein for convenience only and is not to be taken as a limitation on the present invention. The terms “distal” and “proximal” refer, respectively, to directions closer to and away from the insertion tip of a catheter in an implantable catheter assembly. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

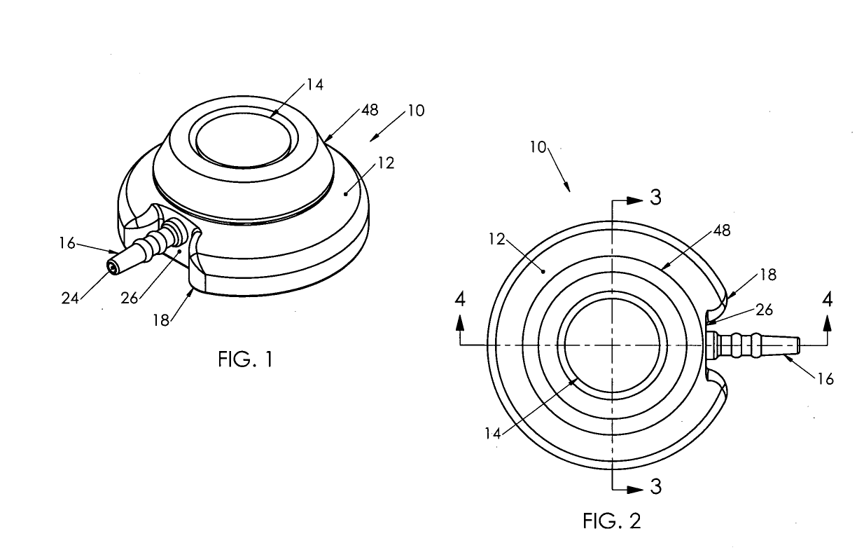

Venous access port assembly 10 of FIGS. 1 to 5 includes a housing 12 and a septum 14, with a discharge port 16 extending from a distal end 18 of the port assembly 10 to be attached securely and sealingly to the proximal end of a catheter (not shown). A passageway 20 extends from the interior reservoir 22 to the distal tip opening 24 of discharge port 16. A recess 26 is seen to be provided along both sides of discharge port 16, facilitating insertion of the discharge port 16 into the catheter lumen and providing a clearance for a locking sleeve or clamp (not shown) utilized to compress the catheter lumen wall against the exterior surface of the discharge port 16 for assured sealed connection of the catheter with the port assembly 10.

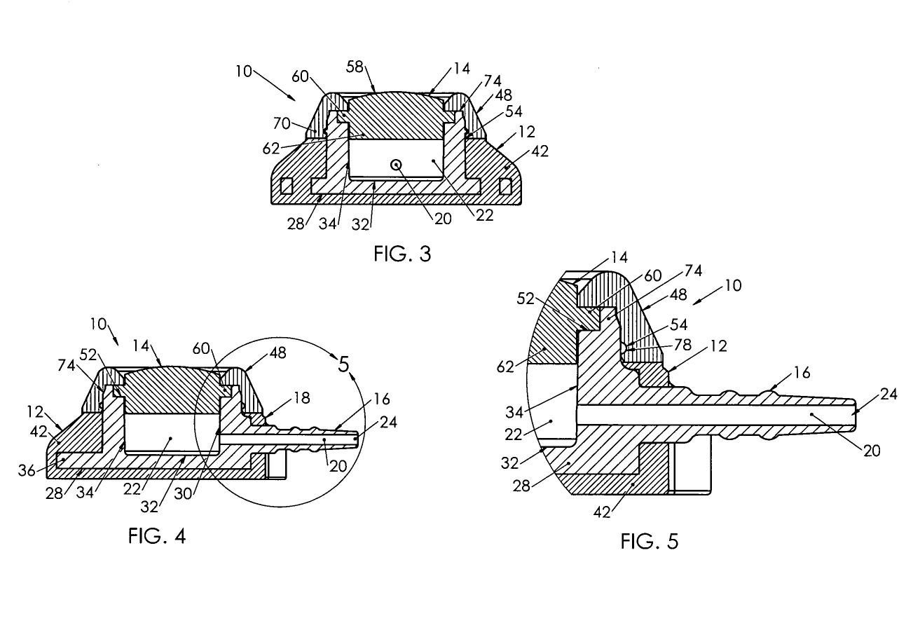

With reference now to FIGS. 3 to 8, the interior of the port assembly 10 is shown to provide an interior reservoir 22. Housing 12 is shown to include a housing base 28 of needle-impenetrable material that includes a well 30 having a bottom floor 32 and side walls 34 that define the interior reservoir 22 beneath septum 14. Bottom floor 32 may be convex or elevated toward the center of the reservoir, if desired. Housing base 28 includes a base flange 36 extending radially outwardly from the bottom of well 30, and base flange 36 includes openings 38, 40 that serve to enable suturing to the patient upon placement of the venous access port and the attached catheter into the patient.

As shown in FIGS. 3 to 5, a skirt 42 is insert molded about housing base 28 and may be of silicone elastomer. It is seen that skirt 42 encapsulates the outer surfaces of the bottom wall 44 and the bottom portion of the side walls 46 of housing base 28, and is shown to fill in the suture holes 38, 40; but since the material is silicone elastomer, suturing is possible since the suturing needle can easily be inserted through the material of skirt 42 and through the suture holes, and thereafter the filled openings provide minimal opportunity for ingrowth of patient tissue into the openings.

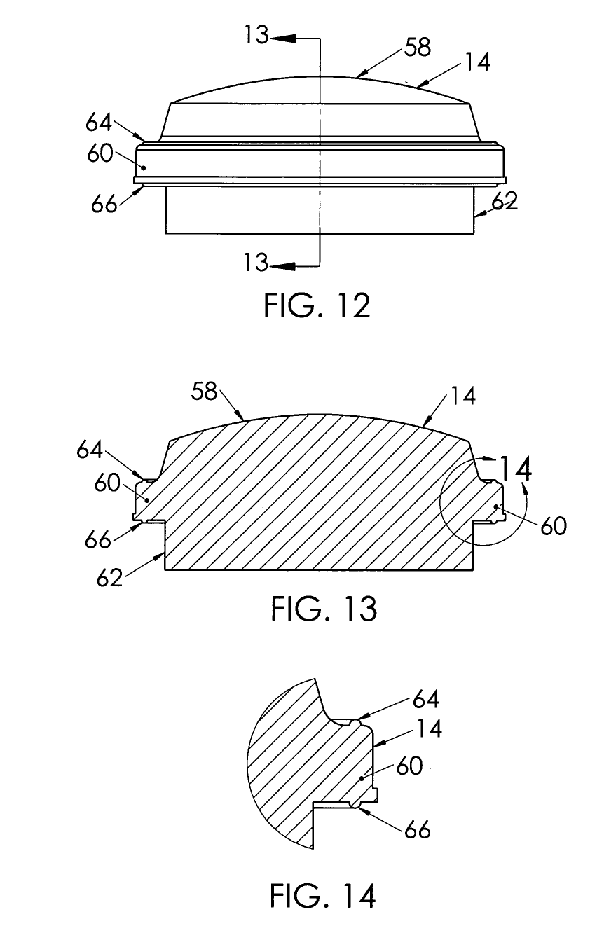

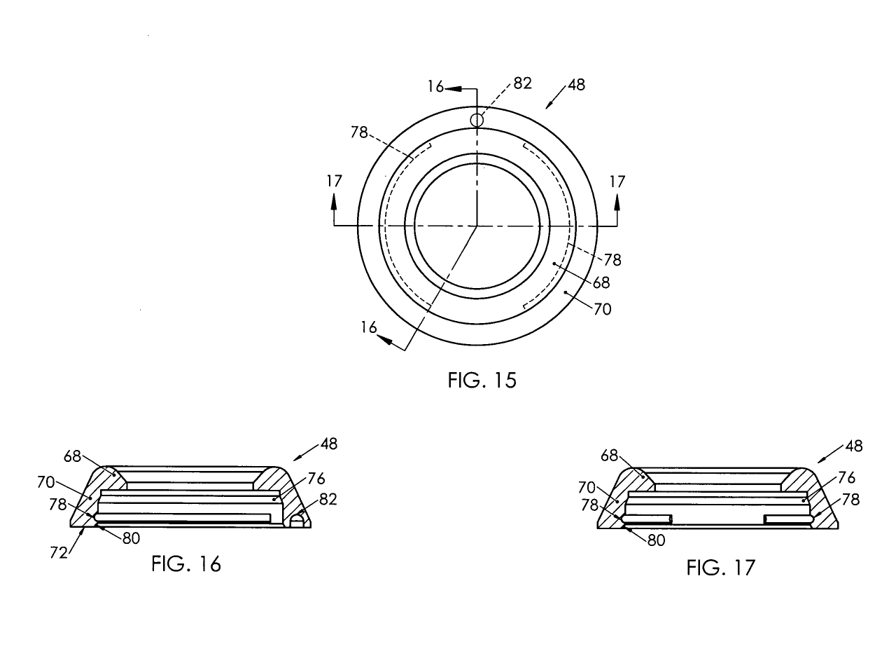

Also seen in FIGS. 1 to 5 is cap 48, which secures to housing base 28 to, in turn, secure septum 14 in position in the port assembly 10. Cap 48 is described in greater particularity with respect to FIGS. 15 to 17, and septum 14 is described in greater particularity with respect to FIGS. 12 to 14. Skirt 42 may be insert molded onto housing base 28 either before or after cap 48 is secured to the upper portion of housing base 28 to secure the septum in position.

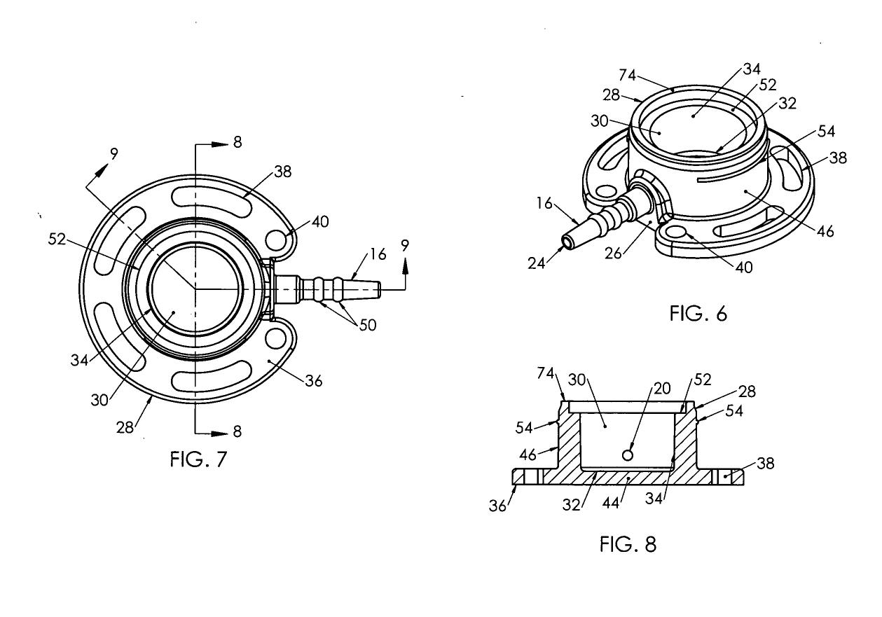

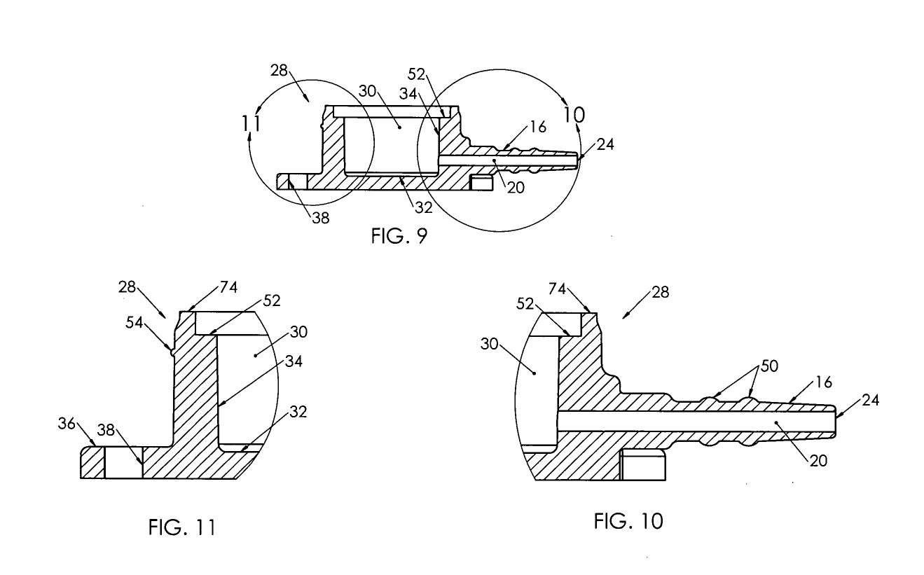

In FIGS. 1, 2, 4 to 7, 9 and 10, it is seen that discharge port 16 is integral with housing base 28; alternatively, the discharge port may comprise a stem component (not shown) such as of metal, that is insert molded to the housing base. Discharge port 16 is shown to have a proximal end connected to the housing, a distal end 24, a passageway 20 therethrough, and a pair of annular ridges 50 that have rounded catheter-engaging surfaces that facilitate the mechanical connection of the catheter proximal end with the port assembly 10. Housing base 28 includes a septum seat 52 extending into the top of well 30, into which a flange of the septum will be seated, preferably under radially inward compression. Also seen in FIGS. 6 and 8 to 11, around the greater part of the side wall 46 of housing base 28 is provided a first retention section, an arcuately extending rounded retention rib 54, which will be discussed in greater detail below.

Septum 14, in FIGS. 12 to 14, is seen to have a rounded dome-shaped top surface 58, an annular seating flange 60, and a plug section 62. The dome-shaped top surface 58 is well-known to medical practitioners as the easily tactilely distinguishable target for needle penetration once the venous access port assembly 10 and the catheter assembled thereto, are implanted within the patient. Seating flange 60 is disposed within flange seat 52 of housing base 28, preferably under radially inward compression. Vertical compression of seating flange 60 is also attained when cap 48 is snapped onto housing base 28, compressing seating flange 60 against flange seat 52, as seen best in FIGS. 3 to 5. Also, seating flange 60 is seen to have rounded ridges 64, 66 disposed on the upper and lower surfaces of seating flange 60 and extending completely around the septum, and are vertically co-aligned. The pair of rounded ridges 64, 66 focuses greater compression circumferentially completely around the upper and lower surfaces of seating flange 60 upon assembly to assure sealing with respect to cap 48 and housing base 28, and sealing the reservoir 22.

Cap 48 is illustrated in FIGS. 15 to 17. Cap 48 includes a top portion 68 that is generously rounded and is preferably dimensioned to extend at least as high as the center of the septum dome 58. Cap 48 also includes an annular side portion 70 that slopes downwardly and radially outwardly to bottom surface 72. Top portion 68 extends radially inwardly to cover both the top edge 74 of housing base 28 and seating flange 60 of septum 14 when assembled. An interior cavity 76 of cap 48 is shaped and dimensioned to receive thereinto both seating flange 60 of septum 14 and the upper portion of side walls 46 of housing base 28. A second retention section or retention groove 78 is provided around the greater part of the side surface of interior cavity 76 that corresponds with and is complementary to the first retention section or retention rib 54 of housing base 28, discussed above with reference to FIGS. 3 to 11. Retention groove 78 and retention rib 54 provide a snap fit, assuredly mechanically securing cap 48 to housing base 28. Preferably, retention rib 54 may have a radius of about 0.010 in (0.254 mm) and retention groove 78 may have a radius of about 0.015 in (0.381 mm). Beneath retention groove 78 is a chamfered surface to facilitate the lower portion of cap 48 being forced over retention rib 54. In addition to the snap fit, a solvent bond is preferably formed between the top portion 74 and adjacent portions of cavity 76 of cap 48, also wicking to adjacent surfaces as would be expected, including the retention rib 54 and retention groove 78.

Preferably, the retention rib 54 of the housing base upper portion comprises two separate coplanar portions evenly spaced apart from each other, each extending circumferentially an angular distance about 90° about the housing base upper portion; and the retention groove 78 of the cap comprises two separate coplanar portions spaced apart from each other, each extending circumferentially an angular distance about 120°. Placing the retention rib on the housing base upper portion and the retention groove on the inner surface of the cap, minimizes the stress on the cap during assembly, since some flexure of the cap is necessary; but, optionally, the retention rib could be positioned on the inner surface of the cap and the related retention groove positioned on the housing base upper portion. Assisting the assembly process, a blind hole 82 is provided on the bottom surface of the cap midway between groove sections, that is easily alignable with the discharge port to assure that the groove sections are angularly aligned with the rib sections for proper snap fit.

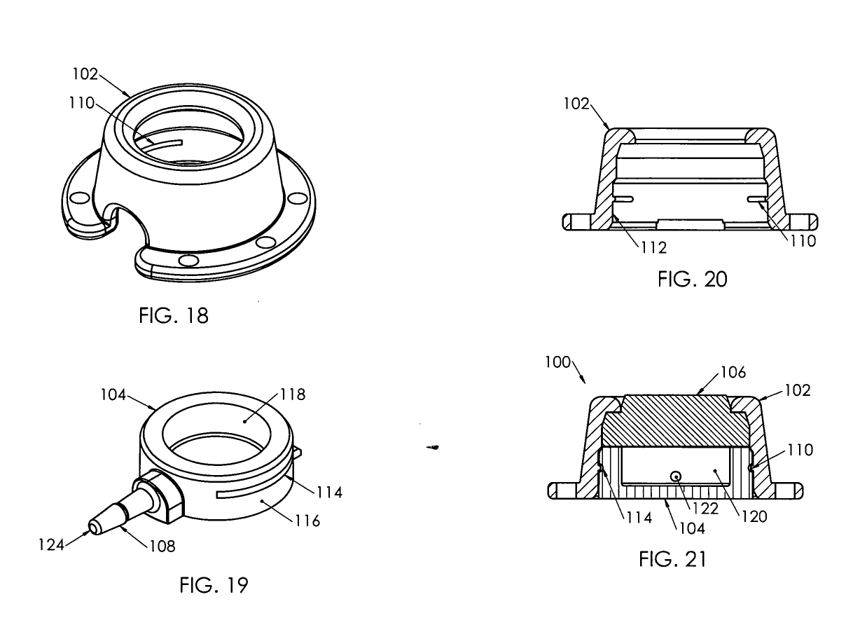

Referring now to FIGS. 18 to 21, a second embodiment of venous access port assembly 100 of the present invention is illustrated. In this embodiment, cap 102 extends downwardly to a bottom edge that is coplanar with the bottom of housing base 104. Septum 106 is secured within the assembly 100 by cap 102 and housing base 104, and housing base 104 includes a discharge port 108. A retention rib 110 protrudes radially inwardly from the inside surface 112 of cap 102, and a retention groove 114 is defined in the exterior surface of the side wall 116 of housing base 104 and complements retention rib 110. It is seen in FIG. 21 that retention rib 110 snaps into retention groove 114 upon complete assembly of the venous access port 100. In this embodiment the retention rib is defined on the cap while the retention groove is defined on the housing base. As with venous access port assembly 10 of FIGS. 1 to 17, the retention groove 114 extends in two spaced portions an angular distance greater than the angular distance of the retention rib 110; in this case the retention rib portions extend respective angular distances of 80° while the retention groove portions extend respective angular distances of 90°. Housing base 104 defines a well 118 that upon assembly forms reservoir 120, and a passageway 122 communicates between reservoir 120 to distal tip opening 124 of discharge port 108.

The advantages of the mechanical retention of the cap to the housing base include assured securement of the cap and the housing base, in addition to the bonding thereof. Another advantage is more efficiency in the manufacturing of the venous access port assembly, and resultant economy: since the septum flange is under compression upon and after assembly, and since bonding is performed to further secure and seal the cap to the housing base, the mechanical lock holds the cap appropriately to the housing base while simultaneously maintaining appropriate compression of the septum flange, thus eliminating the necessity of tooling fixtures that otherwise would be needed to hold the three parts together until the bonding material fully cures.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A venous access port assembly, comprising:
a housing having a discharge port, a needle-penetrable septum and a cap securable to the housing and retaining the septum securely in the assembly,
wherein an upper portion of a housing base of the housing includes a septum seat into which a seating flange of the septum is seated, and
wherein the housing base upper portion includes a first retention section extending circumferentially around much of the upper portion, and the cap includes an interior cavity having an inwardly facing surface associated with and adjacent to the housing base upper portion and a second retention section is defined circumferentially around much of the inwardly facing surface and is associated with the first retention section and complementary thereto such that upon assembly of the cap to the housing base the first and second retention sections snap fit together to assure mechanical joining of the cap to the housing base thereby securing the septum in the housing.

2. The venous access port assembly of claim 1, wherein adjacent surfaces of the cap and the housing base are solvent bonded to each other upon assembly.

3. The venous access port assembly of claim 1, wherein one of the first and second retention sections is a retention rib and the other thereof is a complementary retention groove.

4. The venous access port assembly of claim 3, wherein the retention rib is defined on the outer surface of the housing base and the retention groove is defined on the inwardly facing surface of the interior cavity of the cap.

5. The venous access port assembly of claim 3, wherein the retention rib is defined on the outer surface of the housing base and the retention groove is defined on the inwardly facing surface of the interior cavity of the cap.

6. The venous access port assembly of claim 3, wherein the retention rib comprises two separate coplanar portions spaced apart from each other, each extending circumferentially an angular distance between about 60° to about 100°, and the retention groove comprises two separate coplanar portions spaced apart from each other, each extending circumferentially an angular distance about 70° to about 130° and extending an angular distance greater than that of the complementary retention rib portion.

7. The venous access port assembly of claim 6, wherein the retention rib comprises two separate coplanar portions spaced apart from each other, each extending circumferentially an angular distance between about 80° to about 90°, and the retention groove comprises two separate coplanar portions spaced apart from each other, each extending circumferentially an angular distance about 90° to about 120° and extending an angular distance greater than that of the complementary retention rib portion.

8. A method of assembling a venous access port assembly, comprising the steps of:
providing a port housing base having a well and further having a discharge port and a passageway extending from the well through the discharge part, a needle-penetrable septum, and a cap securable to the housing base to securely retain the septum to the housing base; and
lockably securing the cap to the housing base mechanically.

9. The method of claim 8, further including the step of bonding the cap to the housing base.

10. The method of claim 8, wherein the step of bonding the cap to the housing base occurs when the cap is compressing a septum flange against an opposing surface of the housing base.

11. A discharge port for a medical device for insertion into a catheter end for establishing a sealed fluid connection therewith, comprising:
a stem extending from a proximal end for connection to a medical device, a distal end for insertion into a catheter end, and a passageway therethrough; and
at least one annular protuberance spaced from the distal end, each at least one protuberance having a rounded catheter-engaging outer surface.

12. The discharge port of claim 11, wherein the stem includes two annular protuberances.
